(19) United States
(12) Patent Application Publication
(10) Pub. No.: US 2012/0107082 A1
(43) Pub. Date: May 3, 2012

(54) Climate Cabinet Having Multiple Intake and Output Stations

(75) Inventors: Hubert Heeg, Aschaffenburg (DE); Bernd Czwak, Bruchkoebel (DE); Bernd Schmidt, Altenstadt-Heegheim (DE)

(73) Assignee: THERMO ELECTRON LED GMBH, Langenselbold (DE)

(21) Appl. No.: 13/099,776

(22) Filed: May 3, 2011

(30) Foreign Application Priority Data
May 7, 2010 (DE) 102010019776.9

(51) Int. Cl. B65H 29/00; C12M 1/00; B65G 57/00

(52) U.S. Cl. 414/788.1; 414/217; 435/303.1

(57) ABSTRACT

A climate cabinet is provided. The climate cabinet includes an object storage device, a first intake and output station for the intake and output of objects and an associated first transport device for positioning objects in the object storage device, and has a second intake and output station for the intake and output of objects and an associated second transport device for positioning objects in the object storage device. A system is also provided. The system includes the climate cabinet and multiple workstations, which can each be situated adjacent to the intake and output stations of the climate cabinet, so that the transport devices of the climate cabinet, which are associated with the intake and output stations, can serve the workstations.

### CROSS-REFERENCE TO RELATED APPLICATIONS

This application claims priority to foreign Patent Application DE 10 2010 019776.9, filed on May 7, 2010, the disclosure of which is incorporated herein by reference in its entirety.

### FIELD OF THE INVENTION

The present invention relates to a climate cabinet having multiple intake and output stations.

### BACKGROUND OF THE INVENTION

The invention relates to a climate cabinet. Climate cabinets are typically used for the purpose of storing objects under defined climatic conditions, for example, a defined temperature or in an atmosphere of defined composition. Examples of climate cabinets are heating cabinets, cooling cabinets, or incubators, in which cell cultures or microorganisms are stored at elevated temperature and very high ambient humidity and optionally in an atmosphere enriched using carbon dioxide over a longer period of time. For this purpose, the objects are applied to suitable object carriers. This object carriers are, for example, the known micro-titration plates. At least one object storage device is typically located in the interior of the climate cabinets to receive the object carriers. These object storage devices are either fixed or mounted so they are rotatable (so-called carousel) in the interior.

The interior of a climate cabinet is typically accessible via a door, which is large enough to position or remove an object storage device in or from, respectively, the interior. The provision of a larger door also makes it easier to clean and maintain the interior of a climate cabinet.

An object storage device is typically not charged with object carriers through a larger door, as mentioned above, but rather through a smaller opening or airlock. Such an airlock is adapted in its dimensions to the individual object carriers to be transported through it. Through the use of a smaller opening or airlock, it is possible to keep the climate in the climate cabinet at a comparatively constant level, compared to the charging through a large main door.

The object carriers are typically placed in the interior of the climate cabinet and also removed therefrom through the opening or airlock with the aid of a transport device. Such a transport device is frequently integrated in the climate cabinet and takes over the charging or discharging of the object carriers through the airlock and the transport between airlock and object storage device. In general, one airlock is provided per climate cabinet according to the prior art, the transport between the airlock and an object storage device within the climate cabinet again being performed using a single transport device.

A climate cabinet having at least two intake and output stations is known. Each two intake and output stations are combined into a pair of stations. This pair of intake and output stations is situated or designed in such a way that a single transport device, which is preferably located in the interior of the climate cabinet, can serve both intake and output stations of the pair. In addition, the same transport device also serves the object storage device situated in the interior of the climate cabinet. The provision of intake and output stations in pairs already makes it easier to integrate a climate cabinet in a system having multiple workstations. Nonetheless, it is desirable to implement a simpler integration of a climate cabinet in a system having multiple workstations, which affords greater flexibility with respect to system integration.

### SUMMARY OF THE INVENTION

Embodiments of the present invention advantageously provide a climate cabinet which may be integrated better and more flexibly in a system having multiple workstations. Furthermore, the inventive climate cabinet also allows more rapid handling of objects.

The climate cabinet may be a heating cabinet, a cooling cabinet, an incubator, etc. In one embodiment, the climate cabinet has an object storage device, a first intake and output station for the intake and output of objects, and an associated first transport device for positioning objects in the object storage device, as well as a second intake and output station for the intake and output of objects and an associated second transport device for positioning objects in the object storage device. The object storage device is used for storing objects. These objects can be objects in the solid or liquid phase. For example, they may be chemical substances or biological objects. In particular, they can be cell cultures or microorganisms. These objects are stored in the object storage device. It is possible in particular that the objects are stored on an object carrier, for example, a micro-titration plate, in the object storage device. The objects can also be introduced and stored in the climate cabinet without special object carriers, however. The object storage device itself can be designed in various ways. For example, it can be a fixed or rotatably mounted object storage device. In the latter case, it is referred to as a so-called carousel. It can also be a stacking device (“stacker”), which is generally implemented in the form of a tower. In such a stacking device, various samples or objects can be stored or stacked in various compartments.

The climate cabinet has a first intake and output station for the intake and output of objects and an associated first transport device for positioning objects in the object storage device. Therefore, an assignment occurs between the first intake and output station, on the one hand, and a transport device, on the other hand. The transport device ensures the transport of an intake object through the intake and output station to the object storage device, and also ensures the introduction, e.g., pushing of the object into the object storage device. Furthermore, the same transport device is used for the purpose of transporting the objects back from the object storage device to the intake and output station, the transport device again performing the actual process of removing the object from the object storage device. The transport device can be implemented in one or multiple parts. The intake and output station can be designed in various ways. It can be a simple opening or a complete intake and output station with accessories and/or airlock function. The intake and output station can be permanently integrated in the climate cabinet and is particularly located inside the climate cabinet. However, it is also possible that the intake and output station is located outside the climate cabinet and/or is only fastenable or fastened modularly thereon. The associated transport device can be designed to be provided within the climate cabinet. However, it is also possible that the transport device is located outside the climate cabinet and is integrated in a modular intake and output station provided on the climate cabinet, for example.

A second intake and output station for the intake and output of objects and an associated second transport device for positioning objects in the object storage device are also provided. The first intake and output station may or may not be identical to the second intake and output station. Furthermore, the first transport device and the second transport device may or may not be identical to one another. However, the statements made with respect to the first intake and output station and the associated first transport device otherwise apply to the second intake and output station and to its associated second transport device. Both the first transport device and also the second transport device can serve the object storage device.

The transport devices frequently have a mobile tray, on which the objects to be transported are transported. A transport of the objects can be performed in multiple directions: a transport in all three spatial directions is frequently possible. This is frequently implemented by the use of a lift, which allows raising or lowering of the objects to be transported. This lift system is frequently combined with a system for horizontal displacement, which is used in particular at the moment of the access of the transport device to the object to be transported. For example, a transport device having a tray moves with the aid of the tray under the object to be transported and can receive it. A combination of a displacement in the horizontal direction with a rotation around a vertical axis is frequently also implemented for transport purposes, so that, for example, the positioning of an object in an object storage device can be performed in a different orientation with respect to the climate cabinet than the intake and output of the same object.

According to one embodiment of the invention, the climate cabinet is configured in such a way that the two intake and output stations and their associated transport devices can simultaneously take in and/or output or transport objects. The two transport devices can thus be in operation substantially independently of one another. For example, one object can be loaded simultaneously by the first intake and output station on the first transport device, while another object is simultaneously transferred by the second transport device to the second intake and output station for output purposes. It is also conceivable that the first intake and output station and its associated transport device is occupied with an intake or output of an object, while at the same time the second transport device transports an object within the climate cabinet or already positions it in the object storage device or withdraws it from the object storage device. Such a configuration of the climate cabinet according to the invention allows a chronologically optimized sequence of process steps to be implemented in comparison to the prior art. The two intake and output stations and their associated transport devices thus operate substantially independently of one another and are each chronologically optimized per se.

According to another embodiment of the invention, the climate cabinet is configured in such a way that only one of the transport devices can serve the object storage device at the same time. Notwithstanding this feature, the first and the second transport devices can again operate independently of one another, however. The described configuration is advisable in particular if the object storage device is an object storage device which is situated so it is rotatable in the climate cabinet. In this case, the object storage device will first be put into a rotational position which permits one of the two transport devices to access a selected compartment or a selected position in the object storage device. Subsequently, the object storage device can be rotated into another position, and the access is now possible for the other transport device. A procedure can run in a similar way if the object storage device is situated so it is horizontally displaceable in the climate cabinet. The climate cabinet can also be configured in the case of an object storage device which is provided fixed in relation to the climate cabinet in such a way that only one of the transport devices can serve the object storage device at a time. This prevents possible collisions of the transport devices with one another during access to the object storage device.

According to another embodiment of the invention, the transport devices have an apparatus for the vertical transport of objects. This can be implemented via the above-mentioned lift system, for example. This is advantageous in particular if the objects to be stored in the climate cabinet are stacked one over another, for example, stored in a stacking device.

According to another embodiment of the invention, the two transport devices are designed in the same way. This means that both transport devices are fundamentally designed via the same transport capabilities with respect to the transport directions and with respect to the objects to be transported therewith. With respect to the directions, the transport devices do not necessarily have to be able to operate in exactly the same directions in relation to the climate cabinet, instead this means that the two transport devices have the same number and type of movement capabilities. For example, both transport devices can each have a lift function, a rotation function, and a displacement function in the horizontal.

According to an alternative embodiment of the invention, the two transport devices are designed in different ways. This can relate both to the directions of the transport and also to the possibilities with respect to the objects to be transported. For example, one of the two transport devices can have an additional displacement function, which the other transport device does not have. For example, it is also possible that one transport device can transport larger objects than the other transport device. The selective design of the transport device in the same way or in different ways allows a better individual adaptation of the climate cabinet according to the invention to the respective user requirements and ensures better and more individual system integration of the climate cabinet in a laboratory environment.

According to another embodiment of the invention, the two intake and output stations of the climate cabinet are situated on different sides of the climate cabinet. The two intake and output stations can be situated on adjacent sides of the climate cabinet or also on diametrically opposing sides of the climate cabinet, for example. For example, it is possible that the first intake and output station is situated in a side wall of the climate cabinet, and the second intake and output station is located in a front face of the climate cabinet. Alternatively, it is also possible that the first intake and output station is located in a side wall of the climate cabinet, and the second intake and output station is located on the rear side of the climate cabinet. It is also possible that the first intake and output station is located on a first lateral face, for example, on the left of the climate cabinet, and the second intake and output station is located on the other lateral face, for example, on the right. In all cases, the two intake and output stations can be located at approximately the same height in relation to a floor surface of the climate cabinet. However, it is also possible that the two intake and output stations are located at different heights in relation to a floor surface of the climate cabinet. Of course, it is also possible that the two intake and output stations are not situated on different sides of the climate cabinet, but rather on the same side of the climate cabinet. The intake and output stations can be located centrally in the middle in relation to a lateral face of the climate cabinet or laterally offset to this middle.

According to another embodiment of the invention, the climate cabinet furthermore has a door for removing the object storage device. This door is therefore significantly larger in its dimensions than the two intake and output stations for the intake and output of individual objects. According to one embodiment of the invention, an intake and output station can also be located inside the mentioned door or integrated therein.

Of course, it is also possible that in addition to the first and second intake and output stations having the associated first or second transport device, respectively, a third intake and output station having an associated third transport device for positioning objects in the object storage device is also provided. The third transport device again differs from both the first and also the second transport device. A fourth or further intake and output station including transport device can also be provided in a similar way.

One embodiment of the system includes a climate cabinet, as described above, and multiple workstations, which are situated adjacent to the intake and output stations of the climate cabinet, so that the transport devices of the climate cabinet associated with the intake and output station can also serve the workstations. For example, the transport devices can thus transport objects to the workstations and place them therein and also remove objects from the workstations. The workstations can differ from one another with respect to their functions, but they may also exert the same function. For example, a dispenser device or filling device (“dispenser”), a detection device (“reader”), a washing device (“washer”), a shaking device or stirring device (“shaker”), a sealing device (“sealer”), or a simple provision device for laboratory materials or objects to be handled come into consideration as the workstations.

According to another embodiment of the invention, the climate cabinet is configured to control the system workstations. In this way, the climate cabinet can be combined very simply with various workstations, and system integration is made significantly easier. Control of the entire laboratory system including the workstations can thus be achieved via the climate cabinet.

According to another embodiment of the invention, the climate cabinet has an internal control unit for controlling the climate cabinet itself and the workstations. This means that the control unit is physically located inside the climate cabinet. Alternatively, however, it is also possible that the climate cabinet has an external interface, via which the climate cabinet and therefore optionally also the workstations are controllable. For example, a connection to a typical computer can be produced via such an external interface, which accesses software, which allows control of the climate cabinet. The software can be permanently installed on the computer, however, it can also be provided as portable, for example, in the form of a CD or DVD or in the form of another data carrier.

The above-described exemplary embodiments of the climate cabinet or the system made of climate cabinet and multiple workstations can be entirely or partially combined with one another.

### BRIEF DESCRIPTION OF THE DRAWINGS

The above-described invention will be explained in greater detail with reference to the appended drawings.

FIG. 1 schematically shows a climate cabinet according to an embodiment of the invention having two intake and output stations and the respective associated transport devices; and

FIG. 2 schematically shows an alternative embodiment of a climate cabinet according to the invention, which is integrated in a system having two workstations.

### DETAILED DESCRIPTION

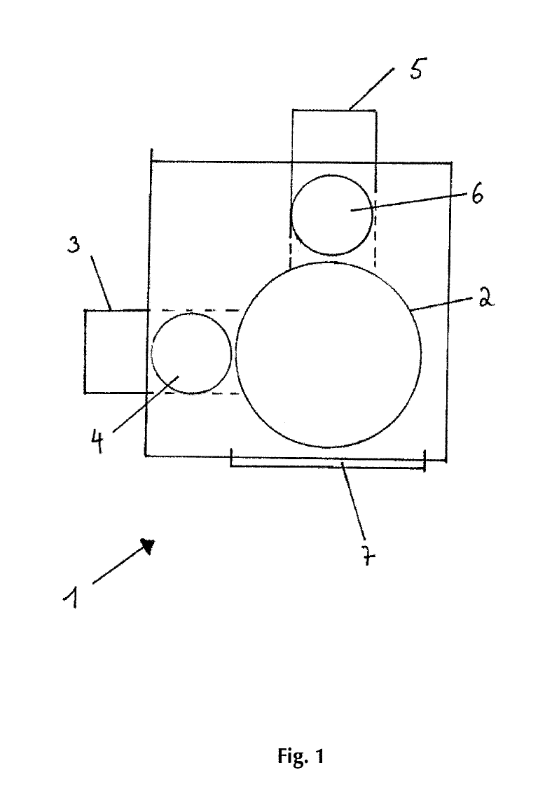

FIG. 1 shows a climate cabinet 1 in a schematic view. This is a schematic top view. The climate cabinet 1 shown is an incubator. An object storage device 2, which can be implemented in the form of a carousel, for example, is located inside the incubator. The object storage device 2 has multiple compartments located in different planes, and is therefore a stacking device. The entire object storage device 2 can be accessed through the large door 7 of the climate cabinet 1.

In operation of the incubator 1, objects are taken in and output through the two intake and output stations 3 and 5. The two stations 3 and 5 are situated on different sides of the climate cabinet 1. In the example shown, they are located on adjacent sides. In the example shown, the intake and output stations 3 and 5 are implemented in the form of a small airlock. The intake and output station 3 is associated with a transport device 4. The intake and output station 5 is associated with a second transport device 6. The first transport device 4 ensures the intake and output of objects through the intake and output station 3 and a transport between the intake and output station 3, on the one hand, and the object storage device 2, on the other hand. Furthermore, the transport device 4 is capable of taking in objects transported thereon directly into the object storage device 2 or removing the objects therefrom. In a similar way, the transport device 6 allows an intake or output of objects through the intake and output station 5 and a transport between the intake and output station 5, on the one hand, and the object storage device 2, on the other hand. Furthermore, a transported object can be put directly into the object storage device 2 or removed therefrom with the aid of the transport device 6. In the exemplary embodiment shown, the two transport devices 4 and 6 are designed essentially identically. They each have a displacement function in a plane, a rotation function, and a lift function, with the aid of which objects to be transported can be transported along an axis which protrudes out of the plane of the paper in FIG. 1. With the aid of this lift function, object storage spaces in the object storage device 2, which are located at various heights, can be reached by the transport devices 4 and 6.

In the exemplary embodiment shown, the climate cabinet 1 is configured in such a way that the two intake and output stations 3 and 5 and their associated transport devices 4 and 6, respectively, can take in or output and/or transport objects simultaneously. The two units made of intake and output station and associated transport device 3 and 4 or 5 and 6 thus operate substantially independently of one another. However, the climate cabinet shown is furthermore configured in such a way that only one of the transport devices 4 or 6 can serve the object storage device 2 at the same time. The rotatably mounted object storage device 2 is thus either brought into a position which allows an access of the transport device 4 to a preselected compartment, or the object storage device 2 is brought into a position which makes an access of the transport device 6 to another preselected compartment or a preselected storage space implementable. This allows a time-saving and also reliable process sequence inside the incubator 1.

The incubator 1 shown in FIG. 1 can be readily integrated into a laboratory system having various workstations. These workstations can be situated close to the intake and output stations 3 or 5 (not shown in FIG. 1).

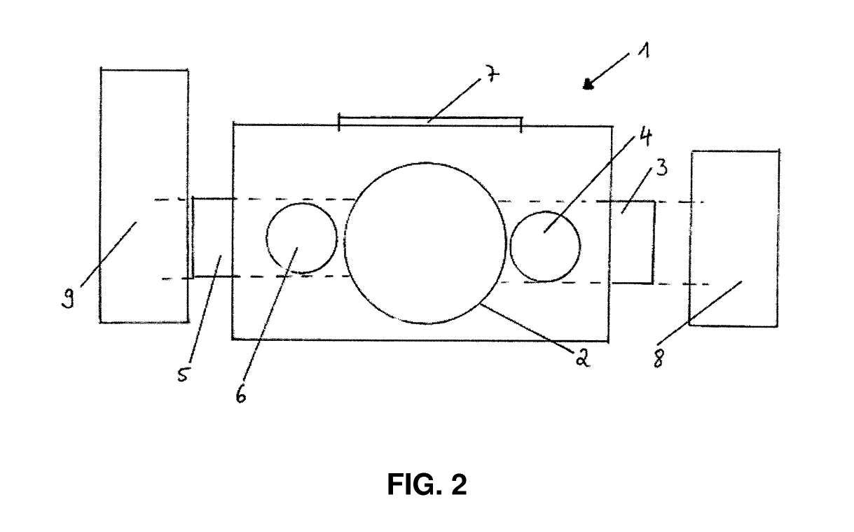

FIG. 2 shows a schematic view of a system having a climate cabinet 1 and multiple workstations 8 and 9. The climate cabinet 1 again has a larger door 7, through which cleaning and maintenance of the climate cabinet 1 and access to the entire object storage device 2 are possible.

In the illustrated exemplary embodiment, the two intake and output stations 3 and 5, which are again designed in the form of an airlock, are situated on diametrically opposing sides of the climate cabinet 1. A transport device 4 or 6 is assigned to each of the intake and output stations 3 or 5, respectively. These transport devices 4 and 6 allow an intake or output of objects through the intake and output stations 3 or 5, a transport between the intake and output stations 3 or 5, on the one hand, and the object storage device 2, on the other hand, and charging or removal of objects into the object storage device 2 or therefrom. The two transport devices 4 and 6 are again designed in a similar way, but they may also be designed differently. In the example shown, both are capable of performing both a horizontal displacement and also a lift movement along an axis Z pointing out of the plane of the paper as well as a rotational movement around its axis Z.

The two workstations 8 and 9 are connected to the two intake and output stations 3 and 5. The workstations are workstations of different types. For example, it is possible that the workstation 9 is an object provision workstation, in which empty objects such as empty micro-titration plates are provided, for example, which are then taken into the climate cabinet 1 through the intake and output station 5 with the aid of the transport device 6. The workstation 8 can be a filling device, for example, in which originally empty objects are filled using a specific material such as a specific solvent or an organic sample, for example. For the filling, for example, empty objects or object carriers can be removed from the object device 2 with the aid of the transport device 4. They are transported with the aid of the transport device 4 to the intake and output station 3 and output through it and provided to the filling device 8. After the filling procedure performed in the filling device 8, the filled objects can be returned with the aid of the transport device 4 through the intake and output station 3 back into the climate cabinet 1 and therein back into the object storage device 2.

In the example shown, the system made of climate cabinet 1 and workstations 8 and 9 is controlled by the climate cabinet 1. For this purpose, the climate cabinet 1 can have an internal control unit for controlling the climate cabinet 1 and the workstations 8 and 9. However, it is also possible that the climate cabinet 1 shown has an external interface, via which the entire climate cabinet 1 is controllable for the purpose of system control.

With the aid of the invention, it is thus possible to integrate a climate cabinet in a simple way in an existing laboratory environment having various workstations. The processing steps performed therein can be executed in a time optimized and individual way.

The many features and advantages of the invention are apparent from the detailed specification, and, thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and, accordingly, all suitable modifications and equivalents may be resorted to that fall within the scope of the invention.

What is claimed is:

1. A climate cabinet, comprising:
an object storage device;
a first intake and output station for intake and output of objects and an associated first transport device for positioning objects in the object storage device; and
a second intake and output station for intake and output of objects and an associated second transport device for positioning objects in the object storage device.

2. The climate cabinet according to claim 1, wherein the first and second intake and output stations are situated on different sides of the climate cabinet.

3. The climate cabinet according to claim 1, wherein the first and second intake and output stations are situated on adjacent sides of the climate cabinet.

4. The climate cabinet according to claim 1, wherein the first and second intake and output stations are situated on diametrically opposing sides of the climate cabinet.

5. The climate cabinet according to claim 1, wherein the object storage device is rotatable in the climate cabinet.

6. The climate cabinet according to claim 1, wherein the object storage device comprises a stacking device.

7. The climate cabinet according to claim 1, wherein the first and second intake and output stations and their associated transport devices take in or output and/or transport objects simultaneously.

8. The climate cabinet according to claim 1, wherein only one of the transport devices serves the object storage device at a time.

9. The climate cabinet according to claim 1, wherein the transport devices include an apparatus for the vertical transport of objects.

10. The climate cabinet according to claim 1, wherein the transport devices have the same design.

11. The climate cabinet according to claim 1, wherein the transport devices have different designs.

12. The climate cabinet according to claim 1, further comprising a control unit for controlling the climate cabinet.

13. The climate cabinet according to claim 1, further comprising a door for removing the object storage device.

14. The climate cabinet according to claim 1, wherein the climate cabinet is an incubator.

15. A system, comprising:
a cabinet according to claim 1; and
a plurality of workstations situated adjacent to the first and second intake and output stations such that the transport devices serve the workstations.

16. The system according to claim 15, wherein the climate cabinet is configured to control the workstations.

17. The system according to claim 16, wherein the climate cabinet has an internal control unit for controlling the climate cabinet and the workstations.

18. The system according to claim 16, wherein the climate cabinet has an external interface via which the climate cabinet is controllable.
